(19) United States
(12) Patent Application Publication
(10) Pub. No.: US 2003/0095944 A1
(43) Pub. Date: May 22, 2003

(54) Conditioner containing particles

(76) Inventor: Sanjeev Midha, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/264,234

(22) Filed: Oct. 3, 2002

Related U.S. Application Data
(60) Provisional application No. 60/328,387, filed on Oct. 10, 2001.
(60) Provisional application No. 60/326,847, filed on Oct. 3, 2001.

(51) Int. Cl. A61K 7/075; A61K 7/08

(52) U.S. Cl. 424/70.27; 424/70.31

(57) ABSTRACT

The compositions of the present invention relate to improved hair care compositions having from about 0.1% to about 10% by weight, of a low melting point fatty alcohol having a melting point of 30° C. or lower; from about 0.01% to about 10% particles; from about 0.1% to about 10% by weight, of a polymer of ethylene oxide, propylene oxide, and mixtures thereof, having the formula:

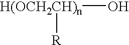

wherein R is selected from the group consisting of H, methyl, and mixtures thereof; and n has an average value of from about 2,000 to about 14,000; from 0% to about 20% by weight, of a conditioning agent selected from the group consisting of cationic surfactants, cationic polymers, nonvolatile silicones, nonvolatile hydrocarbons, saturated C₁₄ to C₂₂ straight chain fatty alcohols, nonvolatile hydrocarbon esters, and mixtures thereof; and from about 50% to about 99.8%, by weight, water.

### CROSS REFERENCE TO RELATED APPLICATION

The application claims the benefit of U.S. Provisional application Serial No. 60/328,387 (Case 8733P2), filed on Oct. 10, 2001 and U.S. Provisional application Serial No. 60/326,847 (Case 8733P), filed on Oct. 3, 2001 in the name of Midha.

### FIELD

The present invention relates to a hair conditioner containing particles. More specifically, it relates to a rinse-off conditioner that deposits particles on the hair.

### BACKGROUND

Solid particles are known for use as benefit agents in a variety of formulations and personal care compositions. Solid particles can impart benefits both to the compositions comprising them or surfaces to which the compositions are applied. Solid particles can for example be used as pigments or coloring agents, opacifiers, pearlescent agents, feel modifiers, oil absorbers, skin protectants, matting agents, friction enhancers, slip agents, conditioning agents, exfoliants, odor absorbers, or cleaning enhancers. Additonionally, many active ingredients useful as treatment agents for various disorders or socially embarrasing conditions are available and typically used in solid particulate form including antiperspirant agents, antidandruff agents, antimicrobials, antibiotics, and sunscreens.

Typically when it is desired to modify the properties of a surface through application of particles, the particles are applied via leave-on preparations which are rubbed, sprayed, or otherwise applied directly onto the surface to be affected. Typical personal care preparations suitable for delivery of solid particles to hair or skin surfaces include as examples moisturizers, lotions, creams, loose or pressed powders, sticks, tonics, gels, and various sprays such as aerosol or pump sprays. These products are typically applied directly to the surface whereupon particles are deposited and retained by the composition itself or by residual non-volatile elements of the composition after evaporation and drying.

It has also been known to formulate solid particle benefit agents into rinse-off or cleansing compositions such as hair rinses, shampoos, liquid and bar soaps, conditioners, or colorants. Frequently the solid particle benefit agent is used to affect the overall appearance, stability or aesthetics of the composition itself. As example, it is well known to add colorant particles, pigments, or pearlescent agents to compositions to improve the acceptability and attractiveness of the product to potential consumers. It is also well known to add particulate benefit agents to affect the in use performance, appearance or aesthetic properties of the composition or to provide a tactile signal to the user. As example, exfoliant particles are frequently used in cleansing compositions to improve abrasion and removal of oils and dirt from washed surfaces and to impart a perceptible “scrubbing” sensation to the user. Typically such solid particle agents are not intended or desired to be deposited onto the substrate and are removed during dilution and rinsing of the composition from the surface to which they are applied.

Given the broad range of benefits which can be delivered through application and retention of solid particles on surfaces, however, it can be highly desireable to have rinse-off compositions capable of depositing an effective level of solid particles to the surface treated with compositions containing the desired solid particle benefit agent. Compositions intended to deposit solid particle benefit agents to hair or skin surfaces are known; however, the efficiency of deposition has heretofore been unacceptable, requiring either an excess of the solid particle agent in the composition to affect delivery or an imperceivable or unacceptable level of the benefit to be obtained. The efficient deposition and retention of solid particle benefit agents is particularly difficult from compositions intended for cleansing or washing of surfaces, such as shampoos or other personal cleansing products, which contain surfactants and other ingredients which are used to solubilize, suspend and remove particle and oily substances from the surfaces treated therewith. It remains, nonetheless, highly desireable to provide the benefits and convenience afforded through deposition of solid particle benefit agents via use of a simple, washing composition.

Cleansing compositions containing cationic polymers to improve deposition of certain conditioning oils, such as silicone oils, capable of imparting conditioning or slip properties to surfaces treated therewith are known. These conditioning oils, however, are limited in the range of physical, optical, and aesthetic benefits they provide. Additionally, it is known that the viscosity, particle size and other factors associated with the conditioning oils can significantly affect their ability to deposit from cleansing compositions. It is also known to include solid particles in compositions containing cationic polymers; however, these particles are often added to modify the appearance or stability of the composition itself and are not deposited along with the conditioning oils or cationic polymers to the surface treated therewith. When deposition of solid particle benefit agents from washing compositions is intended, the compositions available heretofore have suffered from the drawbacks of inefficient deposition requiring use of excess amounts of the particle agent or ineffective benefit delivery. It has also been attempted to make specific modifications to solid particle benefit agents to improve their deposition efficiency or retention from rinse-off compositions; however this approach can negatively impact the inherent properties, availability, utility, and cost of the solid particle benefit agents to be used.

It remains, therefore, highly desireable to have a rinse-off composition, preferably a cleansing composition, capable of containing and effectively depositing and retaining solid particle benefit agents on the surface treated therewith. It has now been discovered that select cationic polymers, when used in the cleansing compositions of the present invention, can surprisingly enhance the deposition and retention of solid particle benefit agents on the surfaces treated therewith.

### SUMMARY

A first embodiment of the present invention is directed to a hair care composition comprising:

(a) from about 0.1% to about 10% by weight, of a low melting point fatty alcohol having a melting point of 30° C. or lower;

(b) from about 0.01% to about 10% particles;

(c) from about 0.1% to about 10% by weight, of a polymer of ethylene oxide, propylene oxide, and mixtures thereof, having the formula:

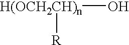

wherein R is selected from the group consisting of H, methyl, and mixtures thereof; and n has an average value of from about 2,000 to about 14,000;

(d) from 0% to about 20% by weight, of a conditioning agent selected from the group consisting of cationic surfactants, cationic polymers, nonvolatile silicones, nonvolatile hydrocarbons, saturated C₁₄ to C₂₂ straight chain fatty alcohols, nonvolatile hydrocarbon esters, and mixtures thereof; and

(e) from about 50% to about 99.8% by weight, water.

A second embodiment of the present invention is directed to a hair care composition comprising by weight:

(a) from about 0.1% to about 10.0% of an amidoamine having the following general formula:

R¹CONH(CH₂)ₘN(R²)₂

wherein R¹ is a fatty acid residue having from about 11 to about 24 carbon atoms, R² is an alkyl having from 1 to about 4 carbon atoms, and m is an integer from 1 to about 4;

(b) from about 0.01% to about 10.0% of an acid; (c) from about 0.05% to about 20% of a high melting point compound having a melting point of at least about 25° C. selected from the group consisting of fatty alcohols, fatty acids, fatty alcohol derivatives, fatty acid derivatives, hydrocarbons, steroids, and mixtures thereof;

(d) from about 0.01% to about 10% particles; and

(e) water;

wherein the molar ratio of the amidoamine to the acid is from about 1:0.3 to about 1:1, and

wherein the composition is substantially free of quaternary ammonium compounds.

A third embodiment of the present invention invention is directed to a hair care composition comprising by weight:

a) from about 0.01% to about 10% hollow particles;

b) from about 0.1% to about 10% of a cationic surfactant; and

c) water.

The present invention is further directed to a method of using the conditioner composition.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

In a first embodiment, the conditioner compositions of the present invention include a low melting point fatty alcohol, particles, a polymer of ethylene oxide, propylene oxide, and mixtures thereof, a conditioning agent and water. In a second embodiment, the conditioner compositions of the present invention include amidoamine, acid, a high melting point compound, particles and water. In a third embodiment, the conditioner compositions of the present invention include particles, a cationic surfactant and water. Each of these components, as well as preferred or optional components, are described in detail hereinafter.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified.

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “fluid” as used herein, means a liquid or a gas which tends to take the shape of its container, container being the wall of the flexible hollow particles.

The term “flexible” as used herein, means that the hollow particles of the present invention are easy to compress but when pressure is reduced the hollow particles regain their original volume.

The term “fluid-encapsulated” as used herein, means that the hollow particles of the invention are structurally hollow. In accordance with the invention, the term “structurally hollow” nonetheless allows the hollow particles to contain at least one additional material therein.

The term “hollow” as used herein, means a particle having an encapsulated area that is substantially free of solid mass, the encapsulated area comprising from 10 to 99.8 percent of the total volume of the particle.

The term “permeable” as used herein, means that a substance that permits a liquid or gas to pass through it under given conditions.

The term “polymer” as used herein shall include materials whether made by polymerization of one type of monomer or made by two (i.e., copolymers) or more types of monomers.

The term “solid” as used herein means a particle that is substantially free of voids.

The term “sphere” as used herein, means a spherical body which is the set of points in a metric space whose distance from a fixed point is approximately constant. Here, the meaning of “approximately” is that the fixed points are within a distance of ±15%.

The term “suitable for application to human hair” as used herein, means that the compositions or components thereof so described are suitable for use in contact with human hair and the scalp and skin without undue toxicity, incompatibility, instability, allergic response, and the like.

The term “water soluble” as used herein, means that the polymer is soluble in water in the present composition. In general, the polymer should be soluble at 25° C. at a concentration of 0.1% by weight of the water solvent, preferably at 1%, more preferably at 5%, most preferably at 15%.

All cited references are incorporated herein by reference in their entireties. Citation of any reference is not an admission regarding any determination as to its availability as prior art to the claimed invention.

Nonvolatile, Low Melting Point Fatty Alcohol

In one embodiment, the compositions of the present invention comprise from about 0.1% to about 10%, by weight, preferably from about 0.1% to about 5%, more preferably from about 0.25% to about 1%, of a nonvolatile low melting point fatty alcohol.

The fatty alcohols hereof have a melting point of 30° C. or less, preferably about 25° C. or less, more preferably about 22° C. or less.

The unsaturated fatty alcohols hereof are also nonvolatile. By nonvolatile what is meant is they have a boiling point at 1.0 atmospheres of at least about 260° C., preferably at least about 275° C., more preferably at least about 300° C.

Suitable fatty alcohols include unsaturated monohydric straight chain fatty alcohols, saturated branched chain fatty alcohols, saturated C₈-C₁₂ straight chain fatty alcohols, and mixtures thereof. The unsaturated straight chain fatty alcohols will typically have one degree of unsaturation. Di- and tri- unsaturated alkenyl chains may be present at low levels, preferably less than about 5% by total weight of the unsaturated straight chain fatty alcohol, more preferably less than about 2%, most preferably less than about 1%.

Preferably, the unsaturated straight chain fatty alcohols will have an aliphatic chain size of from C₁₂-C₂₂, more preferably from C₁₂-C₁₈, most preferably from C₁₆-C₁₈. Especially preferred alcohols of this type include oleyl alcohol and palmitoleic alcohol.

The branched chain alcohols will typically have aliphatic chain sizes of from C₁₂-C₂₂, preferably C₁₄-C₂₀, more preferably C₁₆-C₁₈. Exemplary branched chain alcohols for use herein include isostearyl alcohol, octyl dodecanol, and octyl decanol.

Examples of saturated C₈-C₁₂ straight chain alcohols include octyl alcohol, caprylic alcohol, decyl alcohol, and lauryl alcohol.

The low melting point fatty alcohols hereof are used at a level of from about 0.1% to about 10%, by weight of the composition, more preferably from about 0.1% to about 5%, most preferably from about 0.25% to about 1%.

The present compositions are preferably limited to levels of monohydric saturated straight chain fatty alcohols, such as cetyl alcohol and stearyl alcohol, and other waxy fatty alcohols having melting points above 45° C., of no more than about 5%, by weight of the composition, preferably no more than about 4% since the presence of such waxy fatty alcohols can adversely affect the shine benefits of the present invention. However, it may be desirable to use waxy fatty alcohols for their conditioning benefits.

Particles

The compositions of the present invention include particles. Water insoluble solid particles of various shapes and densities are useful. In a preferred embodiment, the particles tend to have a spherical, an oval, an irregular, or any other shape in which the ratio of the largest dimension to the smallest dimension (defined as the aspect ratio) is less than 10. More preferably, the aspect ratio of the particles is less than 8. Still more preferably, the aspect ratio of the particles is less than 5.

However, it has been found that particles with an aspect ratio of greater than 10 are also useful as long as they remain as aggregated particle stacks or as individual particle stacks on inclusion in an aqueous conditioner composition. Non limiting examples of such particles are Laponite SCPX-2549 and Gelwhite H NF from Southern Clay Products Inc., Flamenco Ultra Silk 2500 and Timica Silkwhite 110W from Engelehard Corp.

Particles useful in the present invention can be natural, synthetic, or semi-synthetic in composition. Hybrid particles are also useful. Synthetic particles can made of either cross-linked or non cross-linked polymers. The particles of the present invention can have surface charges or their surface can be modified with organic or inorganic materials such as surfactants, polymers, and inorganic materials. Particle complexes are also useful.

Non limiting examples of natural particles include various precipitated silica particles in hydrophilic and hydrophobic forms available from Degussa-Huls under the trade name Sipernet. Snowtex colloidal silica particles available from Nissan Chemical America Corporation.

Examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Other examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

In one embodiment of the present invention, the particles used in the conditioner composition are hollow particles. In a preferred embodiment, the hollow particles are fluid-encapsulated, flexible microspheres. The microspheres are structurally hollow, however, they may contain various fluids, which encompass liquids and gases and their isomers. The gases include, but not limited to, butane, pentane, air, nitrogen, oxygen, carbon dioxide, and dimethyl ether. If used, liquids may only partially fill the microspheres. The liquids include water and any compatible solvent. The liquids may also contain vitamins, amino acids, proteins and protein derivatives, herbal extracts, pigments, dyes, antimicrobial agents, chelating agents, UV absorbers, optical brighteners, silicone compounds, perfumes, humectants which are generally water soluble, additional conditioning agents which are generally water insoluble, and mixtures thereof. In one embodiment, water soluble components are preferred encompassed material. In another embodiment, components selected from the group consisting of vitamins, amino acids, proteins, protein derivatives, herbal extracts, and mixtures thereof are preferred encompassed material. In yet another embodiment, components selected from the group consisting of vitamin E, pantothenyl ethyl ether, panthenol, Polygonum multiflori extracts, and mixtures thereof are preferred encompassed material.

The particles of the present invention can have surface charges or their surface can be modified with organic or inorganic materials such as surfactants, polymers, and inorganic materials. Particle complexes are also useful. Non-limiting examples of complexes of gas-encapsulated microspheres are DSPCS-12™ (silica modified ethylene/methacrylate copolymer microsphere) and SPCAT-12™ (talc modified ethylene/methacrylate copolymer microsphere). Both of these are available from Kobo Products, Inc.

The surface of the particle may be charged through a static development or with the attachment of various ionic groups directly or linked via short, long or branched alkyl groups. The surface charge can be anionic, cationic, zwitterionic or amphoteric in nature.

The wall of the particles of the present invention may be formed from a thermoplastic material. The thermoplastic material may be a polymer or copolymer of at least one monomer selected from the following groups: acrylates, methacrylates, styrene, substituted styrene, unsaturated dihalides, acrylonitriles, methacrylonitrile. The thermoplastic materials may contain amide, ester, urethane, urea, ether, carbonate, acetal, sulfide, phosphate, phosphonate ester, and siloxane linkages. The hollow particles may comprise from 1% to 60% of recurring structural units derived from vinylidene chloride, from 20% to 90% of recurring structural units derived from acrylonitrile and from 1% to 50% of recurring structural units derived from a (meth)acrylic monomer, the sum of the percentages (by weight) being equal to 100. The (meth)acrylic monomer is, for example, a methyl acrylate or methacrylate, and especially the methacrylate. Preferably, the particles are comprised of a polymer or copolymer of at least one monomer selected from expanded or non-expanded vinylidene chloride, acrylic, styrene, and (meth)acrylonitrile. More preferably, the particles are comprised of a copolymer of acrylonitrile and methacrylonitrile.

Particles comprised of polymers and copolymers obtained from esters, such as, for example, vinyl acetate or lactate, or acids, such as, for example, itaconic, citraconic, maleic or fumaric acids may also be used. See, in this regard, Japanese Patent Application No. JP-A-2-112304, the full disclosure of which is incorporated herein by reference.

Non-limiting examples of commercially available suitable particles are 551 DE (particle size range of approximately 30-50 μm and density of approximately 42 kg/m³), 551 DE 20 (particle size range of approximately 15-25 μm and density of approximately 60 kg/m³), 461 DE (particle size range of approximately 20-40 μm and density 60 kg/m³), 551 DE 80 (particle size of approximately 50-80 μm and density of approximately 42 kg/m³ ), 091 DE (particle size range of approximately 35-55 μm and density of approximately 30 kg/m³), all of which are marketed under the trademark EXPANCEL™ by Akzo Nobel. Other examples of suitable particles for use herein are marketed under the trademarks DUALITE® and MICROPEARL™ series of microspheres from Pierce & Stevens Corporation. Particularly preferred hollow particles are 091 DE and 551DE 50. The hollow particles of the present invention exist in either dry or hydrated state. The aforesaid particles are nontoxic and non irritating to the skin.

Hollow particles that are useful in the invention can be prepared, for example, via the processes described in EP-56,219, EP-348,372, EP486,080, EP-320,473, EP-112,807 and U.S. Pat. No. 3,615,972, the full disclosure of each of which is incorporated herein by reference.

Alternatively, the wall of the hollow particles useful in the present invention may be formed from an inorganic material. The inorganic material may be a silica, a soda-lime-borosilicate glass, a silica-alumina ceramic, or an alkali alumino silicate ceramic. Non-limiting examples of commercially available suitable low density, inorganic particles are H50/10,000 EPX (particle size range approximately 20-60 μm), S38 (particle size range approximately 15-65 μm), W-210 (particle size range approximately 1-12 μm), W410 (particle size range approximately 1-24 μm), W-610 (particle size range approximately 140 μm), G-200 (particle size range approximately 1-12 μm), G400 (particle size range approximately 1-24 μm), G-600 (particle size range approximately 1-40 μm), all of which are marketed under the trademarks 3M™ Scotchlite™ Glass Bubbles, 3M™ Zeeospheres™ ceramic microspheres, and 3M™ Z-Light Spheres™ Ceramic Microspheres. Also useful are Silica shells (average particle size 3 μm) available from KOBO Products and LUXSILT™ (3-13 μm mean diameter) available from PQ Corporation.

Preferably, the particles of the present invention have a particle size of less than 100 μm. More preferably, the particles of the present invention have a particle size of less than 80 μm. Still more preferably, the particles of the present invention have a particle size of less than 60 μm.

The particles of the present invention preferably have a particle size of 0.1 μm or greater. Preferably, the particles have a particle size of greater than about 0.5 μm. More preferably, the particles of the present invention have a particle size greater than 1 μm. Still more preferably, the particles of the present invention have a particle size greater than 2 μm Even more preferably, the particles of the present invention have a particle size greater than about 3 μm. Still more preferably, the particles of the present invention have a particle size greater than about 4 μm.

Preferably, the particles range from about 1 μm to about 70 μm, still more preferably from about 2 μm to about 65 μm, and even more preferably from about 2 μm to about 60 μm in diameter.

Preferably, the wall of the hollow particles useful in the invention are flexible. “Flexible”, as used herein, means that the hollow particles are easy to compress. When pressure is reduced the hollow particles regain their original volume. The flexible hollow particles could alter their shape under an applied stress, or thermal expansion and contraction due to temperature change. Thus, the particles could expand upon heating.

The particles of the invention may be permeable or non-permeable. “Permeable”, as used herein, means that they permit a liquid or gas to pass through them under given conditions. Preferably, a majority of the particles of the present invention will maintain their structural integrity during normal use of the conditioner composition. More preferably, substantially all of the particles maintain their structural integrity during normal use of the conditioner composition.

Preferred particles will also have physical properties which are not significantly affected by typical processing of the composition. Preferably, particles having melting points greater than about 70° C. are used. Still more preferably, particles having a melting point greater than 80° C. are used and most preferrably particles having melting point of greater than about 95° C. are used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The compositions of the present invention comprise at least 0.01% by weight of particles. Preferably, the compositions of the present invention include at least 0.025% by weight of particles, more preferably at least 0.1%, still more preferably at least 0.2%, and even more preferably at least 0.5% by weight of hollow particles. In the compositions of the present invention, it is preferable to incorporate no more than about 20% by weight of particles, more preferably no more than about 10%, still more preferably no more than 5%, and even more preferably no more than 2% by weight of particles.

Water Soluble, Nonionic, Polymers of Ethylene Oxide and Propylene Oxide

In one embodiment, the compositions of the present invention comprise from about 0.1% to about 10%, more preferably from about 0.2% to about 5%, and most preferably from about 0.5% to about 3% of a polymer of ethylene oxide and/or propylene oxide.

The polymers of the present invention are characterized by the general formula:

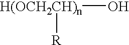

wherein R is selected from the group consisting of H, methyl, and mixtures thereof. When R is H, these materials are polymers of ethylene oxide, which are also known as polyethylene oxides, polyoxyethylenes, and polyethylene glycols.

When R is methyl, these materials are polymers of propylene oxide, which are also known as polypropylene oxides, polyoxypropylenes, and polypropylene glycols. When R is methyl, it is also understood that various positional isomers of the resulting polymers can exist.

In the above structure, n has an average value of from about 2,000 to about 14,000, preferably from about 5,000 to about 9,000, more preferably from about 6,000 to about 8,000.

Polyethylene glycol polymers useful herein that are especially preferred are PEG-2M wherein R equals H and n has an average value of about 2,000 (PEG 2-M is also known as Polyox WSR® N-10 from Union Carbide and as PEG-2,000); PEG-5M wherein R equals H and n has an average value of about 5,000 (PEG 5-M is also known as Polyox WSR® N-35 and Polyox WSR® N-80, both from Union Carbide and as PEG-5,000 and Polyethylene Glycol 300,000); PEG-7M wherein R equals H and n has an average value of about 7,000 (PEG 7-M is also known as Polyox WSR® N-750 from Union Carbide); PEG-9M wherein R equals H and n has an average value of about 9,000 (PEG 9-M is also known as Polyox WSR® N-3333 from Union Carbide); and PEG-14 M wherein R equals H and n has an average value of about 14,000 (PEG 14-M is also known as Polyox WSR® N-3000 from Union Carbide.)

Other useful polymers include the polypropylene glycols and mixed polyethylene/polypropylene glycols.

All percentages describing the polymers in this section of the description herein, are by weight, unless otherwise specified.

Water

The compositions of the present invention comprise water. Preferably, they comprise from about 50% to about 99.8%, by weight, water. The water phase can optionally include other liquid, water-miscible or water-soluble solvents such as lower alkyl alcohols, e.g. C₁-C₅ alkyl monohydric alcohols, preferably C₂-C₃ alkyl alcohols. However, the liquid fatty alcohol must be miscible in the aqueous phase of the composition. Said fatty alcohol can be naturally miscible in the aqueous phase or can be made miscible through the use of cosolvents or surfactants.

The composition of the present invention is an emulsion, having viscosity at 25° C. of at least about 5,000 cP preferably from about 8,000 cP to about 50,000 cP, more preferably from about 15,000 cP to about 35,000 cP. Viscosity is determined by a Brookfield RVT, at 20 RPM.

The compositions of the present invention preferably have a pH of from about 2.5 to about 7, more preferably from about 3 to about 6.8, most preferably from about 3.5 to about 6.5 Higher pH can be utilized as long as the composition retains a viscosity of at least about 8,000 cP at 25° C.

Amidoamine

In another embodiment, the compositions of the present invention comprise by weight from about 0.1% to about 10.0%, preferably from about 0.4% to about 5.0%, more preferably from about 1.0% to about 3.0% of an amidoamine having the following general formula:

R¹CONH(CH₂)ₘN(R²)₂

wherein R¹ is a fatty acid residue having from about 11 to about 24 carbon atoms, R² is an alkyl having from 1 to about 4 carbon atoms, and m is an integer from 1 to about 4.

Preferred amidoamines useful herein include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyidiethylamine, behenamidoethyldimethylamine, arachidamidopropyldimethylamine, arachidamidopropyldiethylamine, arachidamidoethyldiethylamine, arachidamidoethyldimethylamine, and mixtures thereof; more preferably stearamidopropyldimethylamine, stearamidoethyldiethylamine, and mixtures thereof.

Commercially available amidoamines useful herein include: stearamidopropyldimethylamine with tradenames LEXAMINE S-13 available from Inolex (Philadelphia Pa., USA) and AMIDOAMINE MSP available from Nikko (Tokyo, Japan), stearamidoethyldiethylamine with a tradename AMIDOAMINE S available from Nikko, behenamidopropyldimethylamine with a tradename INCROMINE BB available from Croda (North Humberside, England), and various amidoamines with tradenames SCHERCODINE series available from Scher (Clifton N.J., USA).

Acid

In another embodiment, the compositions of the present invention comprise by weight from about 0.01% to about 10.0%, preferably from about 0.05% to about 2.0%, more preferably from about 0.2% to about 1.5%, and still more preferably from about 0.3% to about 1.0%, of an acid. The acid is also included at such a level to provide a molar ratio of the amidoamine to the acid of from about 1:0.3 to about 1:1, preferably from about 1:0.5 to about 1:0.9.

The acid useful herein can be any used by those skilled in the art, including organic acids and inorganic acids. Nonlimiting examples of acids include L-glutamic acid, lactic acid, hydrochloric acid, malic acid, succinic acid, acetic acid, fumaric acid, L-glutamic acid hydrochloride, tartaric acid, and mixtures thereof; preferably L-glutamic acid, lactic acid, hydrochloric acid, and mixtures thereof.

Commercially available acids useful herein are those having the same tradenames as their compound names including: L-GLUTAMIC ACID (cosmetic grade) available from Ajinomoto (Tokyo, Japan), CITRIC ACID (USP) available from Roche (Nutley N.J., USA), ANHYDROUS CITRIC ACID available from Harrmann & Reimer (Springfield N.J., USA), MALIC ACID available from Kyowa Hakko (Tokyo, Japan) and Fuso Kagaku (Osaka, Japan), and lactic acid with a tradename UNICHEM LACA available from UPI.

High Melting Point Compound

In another embodiment, the compositions of the present invention comprise by weight from about 0.05% to about 20%, preferably from about 1% to about 15%, more preferably from about 1.4% to about 10%, still more preferably from about 3% to about 8% of a high melting point compound having a melting point of at least about 25° C. selected from the group consisting of fatty alcohols, fatty acids, fatty alcohol derivatives, fatty acid derivatives, hydrocarbons, steroids, and mixtures thereof. Without being bound by theory, it is believed that these high melting point compounds cover the hair surface and reduce friction, thereby resulting in providing smooth feel on the hair and ease of combing. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992, both of which are incorporated by reference herein in their entirety.

The fatty alcohols useful herein are those having from about 14 to about 30 carbon atoms, preferably from about 16 to about 22 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids can be straight or branched chain acids and can be saturated or unsaturated. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substitued fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

Hydrocarbons useful herein include compounds having at least about 20 carbons.

Steroids useful herein include compounds such as cholesterol.

High melting point compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point compounds useful herein include: cetyl alchol, stearyl alcohol, and behenyl alcohol having tradenames KONOL series available from New Japan Chemical (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy); and cholesterol having tradename NIKKOL AGUASOME LA available from Nikko.

Oily Compound

In another embodiment, the compositions of the present invention optionally comprise by weight from about 0.02% to about 2%, preferably from about 0.2% to about 1.5% of an oily compound having a melting point of not more than about 25° C. selected from the group consisting of a first oily compound, a second oily compound, and mixtures thereof. The oily compounds useful herein may be volatile or nonvolatile. Without being bound by theory, it is believed that, the oily compounds may penetrate the hair to modify the hydroxy bonds of the hair, thereby resulting in providing softness and flexibility to the hair. The oily compound may comprise either the first oily compound or the second oily compound as described herein. Preferably, a mixture of the first oily compound and the second oily compound is used. The oily compounds of this section are to be distinguished from the high melting point compounds described above. Nonlimiting examples of the oily compounds are found in International Cosmetic Ingredient Dictionary, Fifth Edition, 1993, and CTFA Cosmetic Ingredient Handbook, Second Edition, 1992, both of which are incorporated by reference herein in their entirety.

First Oily Compound

A first oily compound may be included in the compositions of the present invention. Preferably the compositions of the present invention comprise by weight from about 0.1% to 0.75% of a first oily compound. The first oily compounds useful herein are selected from the group consisting of fatty alcohols, fatty acids, fatty alcohol derivatives, fatty acid derivatives, and mixtures thereof.

The fatty alcohols useful herein include those having from about 10 to about 30 carbon atoms, preferably from about 12 to about 22 carbon atoms, and more preferably from about 16 to about 22 carbon atoms. These fatty alcohols can be straight or branched chain alcohols and can be saturated or unsaturated alcohols, preferably unsaturated alcohols. Nonlimiting examples of these compounds include oleyl alcohol, palmitoleic alcohol, isostearyl alcohol, isocetyl alchol, undecanol, octyl dodecanol, octyl decanol, octyl alcohol, caprylic alcohol, decyl alcohol and lauryl alcohol.

The fatty acids useful herein include those having from about 10 to about 30 carbon atoms, preferably from about 12 to about 22 carbon atoms, and more preferably from about 16 to about 22 carbon atoms. These fatty acids can be straight or branched chain acids and can be saturated or unsaturated. Suitable fatty acids include, for example, oleic acid, linoleic acid, isostearic acid, linolenic acid, ethyl linolenic acid, ethyl linolenic acid, arachidonic acid, and ricinolic acid.

The fatty acid derivatives and fatty alcohol derivatives are defined herein to include, for example, esters of fatty alcohols, alkoxylated fatty alcohols, alkyl ethers of fatty alcohols, alkyl ethers of alkoxylated fatty alcohols, and mixtures thereof. Nonlimiting examples of fatty acid derivatives and fatty alcohol derivatives, include, for example, methyl linoleate, ethyl linoleate, isopropyl linoleate, isodecyl oleate, isopropyl oleate, ethyl oleate, octyldodecyl oleate, oleyl oleate, decyl oleate, butyl oleate, methyl oleate, octyldodecyl stearate, octyldodecyl isostearate, octyldodecyl isopalmitate, octyl isopelargonate, octyl pelargonate, hexyl isostearate, isopropyl isostearate, isodecyl isononanoate, Oleth-2, pentaerythritol tetraoleate, pentaerythritol tetraisostearate, trimethylolpropane trioleate, and trimethylolpropane triisostearate.

Commercially available first oily compounds useful herein include: oleyl alcohol with tradename UNJECOL 90BHR available from New Japan Chemical, pentaerythritol tetraisostearate and trimethylolpropane triisostearate with tradenames KAKPTI and KAKTTI available from Kokyu Alcohol (Chiba, Japan), pentaerythritol tetraoleate having the same tradename as the compound name available from New Japan Chemical, trimethylolpropane trioleate with a tradename ENUJERUBU TP3SO available from New Japan Chemical, various liquid esters with tradenames SCHERCEMOL series available from Scher, and hexyl isostearate with a tradename HIS and isopropryl isostearate having a tradename ZPIS available from Kokyu Alcohol.

Second Oily Compound

A second oily compound may be included in the compositions of the present invention. Preferably the compositions of the present invention comprise by weight from about 0.1% to about 0.75% of a second oily compound. The second oily compounds useful herein include straight chain, cyclic, and branched chain hydrocarbons which can be either saturated or unsaturated, so long as they have a melting point of not more than about 25° C. These hydrocarbons have from about 12 to about 40 carbon atoms, preferably from about 12 to about 30 carbon atoms, and preferably from about 12 to about 22 carbon atoms. Also encompassed herein are polymeric hydrocarbons of alkenyl monomers, such as polymers of C₂₋₆ alkenyl monomers. These polymers can be straight or branched chain polymers. The straight chain polymers will typically be relatively short in length, having a total number of carbon atoms as described above. The branched chain polymers can have substantially higher chain lengths. The number average molecular weight of such materials can vary widely, but will typically be up to about 500, preferably from about 200 to about 400, and more preferably from about 300 to about 350. Also useful herein are the various grades of mineral oils. Mineral oils are liquid mixtures of hydrocarbons that are obtained from petroleum. Specific examples of suitable hydrocarbon materials include paraffin oil, mineral oil, dodecane, isododecane, hexadecane, isohexadecane, eicosene, isoeicosene, tridecane, tetradecane, polybutene, polyisobutene, and mixtures thereof. Preferred for use herein are hydrocarbons selected from the group consisting of mineral oil, isododecane, isohexadecane, polybutene, polyisobutene, and mixtures thereof.

Commercially available second oily compounds useful herein include isododecane, isohexadeance, and isoeicosene with tradenames PERMETHYL 99A, PERMETHYL 101A, and PERMETHYL 1082, available from Presperse (South Plainfield N.J., USA), a copolymer of isobutene and normal butene with tradenames INDOPOL H-100 available from Amoco Chemicals (Chicago Ill., USA), mineral oil with tradename BENOL available from Witco, and isoparaffin with tradename ISOPAR from Exxon Chemical Co. (Houston Tex., USA).

Free of Ouaternary Ammonium Compounds

In another embodiment, the compositions of the present invention are substantially free of quaternary ammonium compounds, which are commonly used in the art. Examples of quaternary ammonium compounds are those of the general formula:

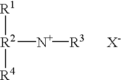

wherein at least one of R¹, R², R³ and R⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R¹, R², R³ and R⁴ are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate, nitrate, sulfate, and alkylsulfate radicals. The aliphatic groups may contain, in addition to carbon and hydrogen atoms, ether linkages, and other groups such as amino groups.

Additional Conditioning Agents

The compositions of the present invention can also comprise one or more additional conditioning agents, such as those selected from the group consisting of cationic surfactants, cationic polymers, nonvolatile silicones (including soluble and insoluble silicones), nonvolatile hydrocarbons, saturated C₁₄ to C₂₂ straight chain fatty alcohols, nonvolatile hydrocarbon esters, and mixtures thereof. Preferred conditioning agents are cationic surfactants, cationic polymers, saturated C₁₄ to C₂₂ straight chain fatty alcohols, and silicones (especially insoluble silicones). The components hereof can comprise from 0% to about 20%, preferably, from about 0.1% to about 20%, more preferably from about 0.5% to about 10%, of additional conditioning agents.

### Cationic Surfactants

Cationic surfactants useful in compositions of the present invention, contain amino or quaternary ammonium moieties. The cationic surfactant will preferably, though not necessarily, be insoluble in the compositions hereof. Cationic surfactants among those useful herein are disclosed in the following documents, all incorporated by reference herein: M.C. Publishing Co., McCutcheon's, Detergents & Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

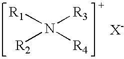

wherein R₁-R₄ are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate nitrate, sulfate, and alkylsulfate radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated. Especially preferred are di-long chain (e.g., di C₁₂-C₂₂, preferably C₁₆-C₁₈, aliphatic, preferably alkyl). di-short chain (e.g., C₁-C₃ alkyl, preferably C₁-C₂ alkyl) quaternary ammonium salts,

Salts of primary, secondary and tertiary fatty amines are also suitable cationic surfactant materials. The alkyl groups of such amines preferably have from about 12 to about 22 carbon atoms, and may be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981, incorporated by reference herein.

Cationic surfactants are preferably utilized at levels of from about 0.1% to about 10%, more preferably from about 0.25% to about 5%, most preferably from about 0.5% to about 2%, by weight of the composition.

### Cationic Polymer Conditioning Agent

The compositions of the present invention can also comprise one or more cationic polymer conditioning agents. The cationic polymer conditioning agents will preferably be water soluble. Cationic polymers are typically used in the same ranges as disclosed above for cationic surfactants.

By “water soluble” cationic polymer, what is meant is a polymer which is sufficiently soluble in water to form a substantially clear solution to the naked eye at a concentration of 0.1% in water (distilled or equivalent) at 25° C. Preferably, the polymer will be sufficiently soluble to form a substantially clear solution at 0.5% concentration, more preferably at 1.0% concentration.

As used herein, the term “polymer” shall include materials whether made by polymerization of one type of monomer or made by two (i.e., copolymers) or more types of monomers.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/gram, more preferably at least about 1.5 meq/gram, even more preferably at least abut 1.1 meq/gram, most preferably at least about 1.2 meq/gram. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain. The average molecular weight of such suitable cationic polymers will generally be between about 10,000 and 10 million, preferably between about 50,000 and about 5 million, more preferably between about 100,000 and about 3 million. Those skilled in the art will recognize that the charge density of amino-containing polymers may vary depending upon pH and the isoelectric point of the amino groups. The charge density should be within the above limits at the pH of intended use.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl meth-acrylamide, wherein the alkyl groups are preferably C₁-C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); co-polymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256, incorporated herein by reference.

Other cationic polymers that can be used include polysaccharide polymers, such as cationic cellulose derivatives and cationic starch derivatives.

Cationic polysaccharide polymer materials suitable for use herein include those of the formula:

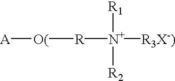

wherein: A is an anhydroglucose residual group, such as a starch or cellulose anhydroglucose residual, R is an alkylene oxyalkylene, polyoxyalkylene, or hydroxyalkylene group, or combination thereof, R₁, R₂, and R₃ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R₁, R₂ and R₃) preferably being about 20 or less, and X is an anionic counterion, as previously described.

Cationic cellulose is available from Amerchol Corp. (Edison, N.J., USA) in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted opoxide, referred to in the industry (CTFA) as Polyquatemium 24. These materials are available from Amerchol Corp. (Edison, N.J., USA) under the tradename Polymer LM-200®.

Other cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418, incorporated by reference herein), and copolymers of etherified cellulose and starch (e.g., as described in U.S. Pat. No. 3,958,581, incorporated herein by reference.)

As discussed above, the cationic polymer hereof is water soluble. This does not mean, however, that it must be soluble in the composition. Preferably however, the cationic polymer is either soluble in the composition, or in a complex coacervate phase in the composition formed by the cationic polymer and anionic material. Complex coacervates of the cationic polymer can be formed with anionic surfactants or with anionic polymers that can optionally be added to the compositions hereof (e.g., sodium polystyrene sulfonate).

### Silicone Conditioning Agents

The compositions hereof can also include nonvolatile soluble or insoluble silicone conditioning agents. By soluble what is meant is that the silicone conditioning agent is miscible with the aqueous carrier of the composition so as to form part of the same phase. By insoluble what is meant is that the silicone forms a separate, discontinuous phase from the aqueous carrier, such as in the form of an emulsion or a suspension of droplets of the silicone.

The silicone hair conditioning agent will be used in the compositions hereof at levels of from about 0.05% to about 10% by weight of the composition, preferably from about 0.1% to about 6%, more preferably from about 0.5% to about 5%, most preferably from about 0.5% to about 3%.

Soluble silicones include silicone copolyols, such as dimethicone copolyols, e.g. polyether siloxane-modified polymers, such as polypropylene oxide, polyethylene oxide modified polydimethylsiloxane, wherein the level of ethylene and/or propylene oxide sufficient to allow solubility in the composition.

Preferred, however, are insoluble silicones. The insoluble silicone hair conditioning agent for use herein will preferably have viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Corning Corporate Test Method CTM0004, Jul. 20, 1970.

Suitable insoluble, nonvolatile silicone fluids include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. The term “nonvolatile” as used herein shall mean that the silicone has a boiling point of at least about 260° C., preferably at least about 275° C., more preferably at least about 300° C. Such materials exhibit very low or no significant vapor pressure at ambient conditions. The term “silicone fluid” shall mean flowable silicone materials having a viscosity of less than 1,000,000 centistokes at 25° C. Generally, the viscosity of the fluid will be between about 5 and 1,000,000 centistokes at 25° C., preferably between about 10 and about 300,000 centistokes.

Silicone fluids hereof also include polyalkyl or polyaryl siloxanes with the following structure:

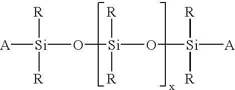

wherein R is alkyl or aryl, and x is an integer from about 7 to about 8,000 may be used. “A” represents groups which block the ends of the silicone chains.

The alkyl or aryl groups substituted on the siloxane chain (R) or at the ends of the siloxane chains (A) may have any structure as long as the resulting silicones remain fluid at room temperature, are hydrophobic, are neither irritating, toxic nor otherwise harmful when applied to the hair, are compatible with the other components of the composition, are chemically stable under normal use and storage conditions, and are capable of being deposited on and of conditioning hair.

Suitable A groups include methyl, methoxy, ethoxy, propoxy, and aryloxy. The two R groups on the silicone atom may represent the same group or different groups. Preferably, the two R groups represent the same group. Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred.

The nonvolatile polyalkylsiloxane fluids that may be used include, for example, polydimethylsiloxanes. These siloxanes are available, for example, from the General Electric Company in their ViscasilR and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

Especially preferred, for enhancing the shine characteristics of hair, are highly arylated silicones, such as highly phenylated polyethyl silicone having refractive indices of about 1.46 or higher, especially about 1.52 or higher. When these high refractive index silicones are used, they should be mixed with a spreading agent, such as a surfactant or a silicone resin, as described below to decrease the surface tension and enhance the film forming ability of the material.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level should be sufficiently low to prevent solubility in the composition hereof.

References disclosing suitable silicone fluids include U.S. Pat. No. 2,826,551, Geen; U.S. Pat. No. 3,964,500, Drakoff, issued Jun. 22, 1976; U.S. Pat. No. 4,364,837, Pader; and British Patent 849,433, Woolston. All of these patents are incorporated herein by reference. Also incorporated herein by reference is Silicon Compounds distributed by Petrarch Systems, Inc., 1984. This reference provides an extensive (though not exclusive) listing of suitable silicone fluids.

Another silicone hair conditioning material that can be especially useful in the silicone conditioning agents is insoluble silicone gum. The term “silicone gum”, as used herein, means polyorganosiloxane materials having a viscosity at 25° C. of greater than or equal to 1,000,000 centistokes. Silicone gums are described by Petrarch and others including U.S. Pat. No. 4,152,416, Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference. The “silicone gums” will typically have a mass molecular weight in excess of about 200,000, generally between about 200,000 and about 1,000,000. Specific examples include polydimethylsiloxane, (polydimethylsiloxane) (methylvinylsiloxane) copolymer, poly(di-methylsiloxane) (diphenyl siloxane)(methylvinylsiloxane) copolymer and mixtures thereof.

Preferably the silicone hair conditioning agent comprises a mixture of a polydimethylsiloxane gum, having a viscosity greater than about 1,000,000 centistokes and polydimethylsiloxane fluid having a viscosity of from about 10 centistokes to about 100,000 centistokes, wherein the ratio of gum to fluid is from about 30:70 to about 70:30, preferably from about 40:60 to about 60:40.

An optional ingredient that can be included in the silicone conditioning agent is silicone resin. Silicone resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetrachlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Silicone resins can enhance deposition of silicone on the hair and can enhance the glossiness of hair with high refractive index volumes.

Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989, incorporated herein by reference.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO)_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

Other Ingredients

The compositions herein can contain a variety of other optional components suitable for rendering such compositions more cosmetically or aesthetically acceptable or to provide them with additional usage benefits. Such conventional optional ingredients are well-known to those skilled in the art.

A wide variety of additional ingredients can be formulated into the present composition. These include: other conditioning agents; hair-hold polymers; detersive surfactants such as anionic, nonionic, amphoteric, and zwitterionic surfactants; additional thickening agents and suspending agents such as xanthan gum, guar gum, hydroxyethyl cellulose, methyl cellulose, hydroxyethylcellulose, starch and starch derivatives; viscosity modifiers such as methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Such optional ingredients generally are used individually at levels from about 0.01% to about 10.0%, preferably from about 0.05% to about 5.0% by weight of the composition.

Friction Test

After treatment with the compositions of the present invention, hair will preferably demonstrate a friction coefficient of from about 1 to about 2. More preferably, the hair will demonstrate a friction coefficient of from about 1.05 to about 1.8. Still more preferably, the hair will demonstrate a friction coefficient of from about 1.1 to about 1.7. Even more preferably, the hair will demonstrate a friction coefficient of from about 1.2 to about 1.6. The friction coefficient is determined according to the following method:

The method measures the change in friction of an untreated versus treated hair switch. A weighted “sled” (˜4.6 mm×3.1 mm) weighing 73 g+/−5% and covered with a nylon mesh of approximately 105 micron pore size is attached to a force measurement device such as an Instron and pulled at a constant speed across a 20 g hair switch (approximately 10 inches in length) that is clamped on one end.

The hair that is used is Caucasian hair that has been formed into 20 gram switches that are ten inches in length which has been cleaned to remove any foreign soils. The hair switches are then allowed to equilibrate in a constant temperature room at 75° C. / 50% relative humidity (RH) overnight.

The hair switches are treated as follows: The switches are pre-wetted under the running tap water. The excess water is squeezed out and the switch is hung on a rack. For leave-on products, 1.5 cc of the test product is then applied to the surface of the hair switch and rubbed into the switch for ˜30-40 sec. For rinse-off products, the test product is applied to the wet hair at a dose of 0.1 g/g and massaged/lathered for approximately 30 seconds followed by approximately 30 seconds of water rinsing at a flow rate of approximately 1.5 gallon/minute and the process repeated to complete one treatment cycle. Switches are then dried and the previously described treatment cycle is repeated for a total of 3 cycles. After treatment, the switches are re-hung on the rack and placed in a constant temperature room (75° C./50% RH) to equilibrate overnight.

The switch to be tested is clamped into position on a horizontal testing stand and combed 2-3 times to orient the hair and remove tangles. The “sled” is then attached and placed on the hair switch. Friction is measured on three separate switches in the forward direction (toward tip end of hair) by pulling the “sled” at a rate of approximately 1 cm/sec while measuring the tension force (typically measured in grams). Each force measurement is an average of at least ten values recorded over a distance of at least 5 cm once the “sled” has reached constant speed. A minimum of three measurements are taken for each switch. The friction coefficient is determined as the average ratio of the friction of the treated hair switch divided by the friction of the untreated hair switch.

Hair Feel Test

After treatment with the compositions of the present invention, hair will preferably demonstrate a “hair feel” of less than about 8. More preferably, the hair will demonstrate a “hair feel” of less than about 7. Still more preferably, the hair will demonstrate a “hair feel” of less than about 6. Even more preferably, the hair will demonstrate a “hair feel” of less than about 5. The “hair feel” is determined according to the following method:

Treated hair switches are evaluated by at least 5 panelists trained to feel dry hair switches. 20 gram, 10 inch long, caucasian hair switches are treated as described for the Friction Test described herein with the compositions of the present invention and are evaluated for the amount of coated feel. A 0-10 scale is used to rate the treated hair switches. A “0“rating indicates very clean feeling hair. A “10” rating indicates a very coated feel to the hair. The end points of the scale are based on hair switches treated as follows:

0 =Treated with Pantene™ Clarifying Shampoo

10=0.75 cc of Pemulen TR-1 gel (from B.F. Goodrich) containing 2.0% Silica (Sipernet 22LS, Degussa) particles rubbed in the hair switch for 30 seconds.

Three different hair switches are used per composition to be tested. The panelists evaluate each treated hair switch and the scores are averaged to produce an overall “hair feel” rating.

### METHOD OF USE

The hair care compositions of the present invention are used in conventional ways to provide the conditioning and other benefits of the present invention. Such method of use depends upon the type of composition employed but generally involves application of an effective amount of the product to the hair, which may then be rinsed from the hair (as in the case of hair rinses) or allowed to remain on the hair (as in the case of gels, lotions, and creams). “Effective amount” means an amount sufficient enough to provide a dry combing benefit. In general, from about 1 g to about 50 g is applied to the hair on the scalp. The composition is distributed throughout the hair, typically by rubbing or massaging the hair and scalp. Preferably, the composition is applied to wet or damp hair prior to drying of the hair. After such compositions are applied to the hair, the hair is dried and styled in accordance with the preference of the user. In the alternative, the composition is applied to dry hair, and the hair is then combed or styled in accordance with the preference of the user.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention.

All parts, percentages, and ratios herein are by weight unless otherwise specified. Some components may come from suppliers as dilute solutions. The levels given reflect the weight percent of the active material, unless otherwise specified.

### Examples 1-7

Hair rinse compositions of the present invention are prepared as follows:

| Component | Ex. 1 | Ex. 2 | Ex. 3 | Ex. 4 | Ex. 5 | Ex. 6 | Ex. 7 |
|---|---|---|---|---|---|---|---|
| Oleyl Alcohol | 1.00 | 1.00 | 1.00 | 1.00 | 0.25 | 0.25 | 1.00 |
| PEG-7M(1) | 2.00 | 1.00 | | 1.00 | — | 1.00 | — |
| PEG-2M(2) | — | — | 1.00 | — | 1.00 | | |
| PEG-14M(2) | | | | | | — | 0.25 |
| Polydimethylsiloxane | 4.20 | 4.20 | 4.20 | 4.20 | 4.20 | 4.20 | 4.20 |
| (3) | | | | | | | |
| Silicone Resin(4) | 0.25 | 0.25 | 0.25 | 0.25 | — | 0.25 | 0.25 |
| Pentaphenyl Trimethyl | 0.38 | 0.38 | 0.38 | 0.38 | — | 0.38 | 0.38 |
| Trisiloxane(5) | | | | | | | |
| DL Panthenol | 0.04 | 0.04 | 0.04 | 0.04 | 0.04 | 0.04 | 0.04 |
| Panthenyl Ethyl Ether | 0.34 | 0.34 | 0.34 | 0.34 | 0.34 | 0.34 | 0.34 |
| Fragrance | 0.30 | 0.35 | 0.35 | 0.35 | 0.35 | 0.35 | 0.35 |
| Kathon ™ CG(6) | 0.03 | 0.03 | 0.03 | 0.03 | 0.03 | 0.03 | 0.03 |
| Cetyl Alcohol | 1.20 | 1.80 | 1.80 | 1.20 | 1.20 | 1.80 | 1.80 |
| Stearyl Alcohol | 0.80 | 1.20 | 1.20 | 0.80 | 0.80 | 1.20 | 1.20 |
| Ditallow Dimethyl | 0.75 | 0.75 | 0.75 | 0.75 | 0.75 | 0.75 | 0.75 |
| Ammonium Chloride | | | | | | | |
| Stearamidopropyl | 1.00 | 1.00 | 1.00 | 1.00 | 1.00 | 1.00 | 1.00 |
| Dimethylamine | | | | | | | |
| Glycerol Monostearate | 0.25 | 0.25 | 0.25 | 0.25 | 0.25 | 0.25 | 0.25 |
| Citric Acid | 0.19 | 0.22 | 0.22 | 0.19 | 0.22 | 0.22 | 0.22 |
| Hydroxyethyl | — | 0.25 | 0.25 | — | 0.25 | 0.25 | 0.25 |
| Cellulose | | | | | | | |
| Mineral Oil | — | — | — | — | 0.25 | | |
| Particles | | | | | | | |
| precipitated silica(10) | | | 1.00 | | | | |
| polymethyl- | | 2.00 | | | | | |
| silsesquioxane(11) | | | | | | | |
| polymethyl- | | | 3.00 | 1.00 | | | |
| silsesquioxane(9) | | | | | | | |
| dimethicone/ | | | | 1.00 | | | |
| vinyldimethicone | | | | | | | |
| crosspolymer powder | | | | | | | |
| (12) | | | | | | | |
| polyethylene(13) | 2.00 | | | | | | |
| PTFE(14) | 2.00 | | | | | | |
| Expancel DE091(15) | | | | | 2.00 | | |
| Iron Oxides(16) | | | | | | 5.00 | |
| Flamenco Velvet Pearl | | | | | | | 2.00 |
| (TiO2/mica)(17) | | | | | | | |
| Cloisonne Satin | | | | | | | 2.00 |
| Bronze (iron oxide/ | | | | | | | |
| mica)(18) | | | | | | | |
| Water | q.s | q.s | q.s | q.s | q.s | q.s | q.s |

(1)PEG-7M is Polyethylene Glycol where n has an average value of about 7,000 and is commercially available under the tradename of Polyox WSR N-750 from Union Carbide.
(2)PEG-2M is Polyethylene Glycol where n has an average value of about 2,000 and is commercially available under the tradename of Polyox WSR N-10 from Union Carbide.
(3)An 85%/15% (wt. Basis) mixture of D5 Cyclomethicone and dimethicone gum (weight average molecular weight of about 4000,000 to about 600,000).
(4)Polytrimethyl hydrosilysilicate,m added as a 50 wt. % solution in decamethylcyclopentasiloxane, General Electric Silicone Products, SS 4320.
(5)Dow Corning 705, Dow Corning Corp. (Midland, MI, USA).
(6)Methylchloroisothiazolinc (and) methylisothiazolinc, a preservative from Rohm & Haas Co., (Philadelphia, USA.)
(10)Sipernat 22LS available from Degussa
(11)Tospearl 240 available from GE Silicones
(12)Tospearl 3120 available from GE Silicones
(13)9506 Cosmetic Powder available from Dow Corning
(14)Microsilk 419 available from MicroPowders, Inc.
(15)PTFE-20 polytetrafluoroethylene powder from Presperse, Inc.
(16)available from Expancel Corp.
(17)Red lion Oxide 70101 available from Cardre, Inc.
(18)available from Engelhard, Corp

### Examples 8-15

Hair rinse compositions of the present invention are prepared as follows:

| Component | Ex. 8 | Ex. 9 | Ex. 10 | Ex. 11 | Ex. 12 | Ex. 13 | Ex. 14 | Ex. 15 |
|---|---|---|---|---|---|---|---|---|
| Stearamidopropyldimethylamine(1) | 2.00 | 1.60 | 2.00 | 1.60 | 2.00 | 2.00 | — | — |
| Stearamidoethyldiethylamine(9) | | | | | — | — | 2.00 | 2.00 |
| L-Glutamic Acid(2) | 0.64 | 0.51 | 0.64 | 0.51 | 0.64 | 0.64 | 0.64 | 0.64 |
| Cetyl Alcohol(3) | 2.50 | 2.00 | 2.50 | 5.60 | 7.00 | — | 7.00 | 7.00 |
| Stearyl Alcohol(4) | 4.50 | 3.60 | 4.50 | — | — | 7.00 | — | — |
| Oleyl Alcohol(5) | 0.25 | 0.20 | 0.58 | 0.20 | 0.25 | 0.58 | 0.25 | 0.20 |
| Mineral Oil(6) | 0.25 | 0.20 | 0.58 | 0.20 | 0.25 | 0.58 | 0.25 | 0.40 |
| Silicones(7) | 4.20 | 2.00 | 2.00 | 3.80 | 0.25 | 1.80 | 2.60 | 0.40 |
| Benzyl Alcohol | 0.40 | 0.40 | 0.40 | 0.40 | — | 0.40 | — | — |
| EDTA | 0.10 | 0.10 | 0.10 | 0.10 | 0.10 | 0.10 | — | 0.10 |
| Methyl Paraben | | | | | 0.20 | — | 0.20 | 0.20 |
| Propyl Paraben | | | | | 0.10 | — | 0.10 | 0.10 |
| Kathon CG(8) | 0.03 | 0.03 | 0.03 | 0.03 | 0.03 | 0.03 | 0.03 | 0.03 |
| Sodium Chloride | — | 0.01 | — | 0.01 | | | | |
| Perfume | 0.20 | 0.20 | 0.20 | 0.20 | 0.20 | 0.20 | 0.20 | 0.20 |
| Particles | | | | | | | | |
| precipitated silica(10) | | | 1.00 | | | | | |
| polymethylsilsesquioxane(11) | | 2.00 | | | | | | 1.50 |
| polymethylsilsesquioxane(12) | | | 3.00 | 1.00 | | | | |
| dimethicone/vinyldimethicone | | | | 1.00 | | | | |
| crosspolymer powder(13) | | | | | | | | |
| polyethylene(14) | 2.00 | | | | | | | |
| PTFE(15) | 2.00 | | | | | | | |
| Expancel DE091(16) | | | | | 2.00 | | | 1.50 |
| Iron Oxides(17) | | | | | | 5.00 | | |
| Flamenco Velvet Pearl (TiO2/ | | | | | | | 2.00 | |
| mica)(18) | | | | | | | | |
| Water | qs | qs | qs | qs | qs | qs | qs | qs |

(1)Stearamidopropyldimethylamine: AMIDOAMINE MPS obtained by Nikko
(2)L-glutamic acid: L-GLUTAMIC ACID (cosmetic grade) obtained by Ajinomoto
(3)Cetyl Alcohol: KONOL series obtained by New Japan Chemical
(4)Stearyl Alcohol: KONOL series obtained by New Japan Chemical
(5)Oleyl Alcohol: UNJECOL 90BHR obtained by New Japan Chemical
(6)Mineral Oil: BENOL obtained by Witco
(7)Silicones: 85%/15% (weight base) mixture of D5 Cyclomethicone and dimethicone gum (weight average molecular weight of about 400,000 to about 600,000) obtained by General Electric Co.
(8)Kathon CG: Mixture of methylcholorisothiazoline and methylisothiazoline obtained by Rohm & Hass Co., (Philadelphia, PA, USA).
(9)Stearamidoethyldiethylamine: AMIDOAMINE S obtained by Nikko
(10)Sipernat 22LS available from Degussa
(11)Tospearl 240 available from GE Silicones
(12)Tospearl 3120 available from GE Silicones
(13)9506 Cosmetic Powder available from Dow Corning
(14)Microsilk 419 available from MicroPowders, Inc.
(15)PTFE-20 polytetrafluoroethylene powder from Presperse, Inc.
(16)available from Expancel Corp.
(17)Red Iron Oxide 70101 available from Cardre, Inc.
(18)available from Engelhard, Corp

For examples 8 through 11, water, stearamidopropyldimethylamine and about 50% of L-glutamic acid are mixed at a temperature above 70° C. Then the high melting point fatty compounds and benzyl alcohol are added with agitation. After cooling down below 60° C., the remaining L-glutamic acid and other remaining components are added with agitation, then cooled down to about 30° C. Examples 8 through 11 have many advantages. For example, they can provide richness and creaminess to the wet hair, and also show good combing feel and lustery appearance when the hair is dry.

For examples 12 through 15, water, the amidoamine and 50% of L-glutamic acid are mixed at a temperature above 70° C. Then the high melting point fatty compounds and bezyl alcohol, if applicable, are added with agitation. After cooling down below 60° C., the remaining 50% of L-glutamic acid and remaining components are added with agitation, then cooled down to about 30° C.

It is understood that the examples and embodiments described herein are for illustrative purposes only and that various modifications or changes in light thereof will be suggested to one skilled in the art without departing from the scope of the present invention.

What is claimed is:

1. A hair care composition comprising:
(a) from about 0.1% to about 10% by weight, of a low melting point fatty alcohol having a melting point of 30° C. or lower;
(b) from about 0.01% to about 10% particles;
(c) from about 0.1% to about 10% by weight, of a polymer of ethylene oxide, propylene oxide, and mixtures thereof, having the formula: 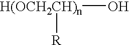
wherein R is selected from the group consisting of H, methyl, and mixtures thereof; and n has an average value of from about 2,000 to about 14,000;
(d) from 0% to about 20% by weight, of a conditioning agent selected from the group consisting of cationic surfactants, cationic polymers, nonvolatile silicones, nonvolatile hydrocarbons, saturated C₁₄ to C₂₂ straight chain fatty alcohols, nonvolatile hydrocarbon esters, and mixtures thereof; and
(e) from about 50% to about 99.8% by weight, water.

2. The composition of claim 1 wherein the low melting point fatty alcohol is selected from the group consisting of unsaturated C₈-C₂₂ straight chain fatty alcohols, saturated C₁₂-C₁₈ branched chain fatty alcohols, saturated C₈-C₁₂ straight chain fatty alcohols, and mixtures thereof.

3. The composition of claim 2 wherein the low melting point fatty alcohol has a melting point of 25° C. or lower.

4. The composition of claim 3 wherein the fatty alcohol is selected from the group consisting of unsaturated C₁₆-C₁₈ straight chain fatty alcohols, C₁₄-C₁₈ branched chain fatty alcohols, and mixtures thereof.

5. The composition of claim 4 wherein the fatty alcohol is oleyl alcohol.

6. The composition of claim 1 wherein n has an average value of from about 5,000 to about 9,000.

7. The composition of claim 6 wherein the low melting point fatty alcohol is selected from the group consisting of unsaturated C₈-C₂₂ straight chain fatty alcohols, saturated C₁₂-C₁₈ branched chain fatty alcohols, saturated C₈-C₁₂ straight chain fatty alcohols, and mixtures thereof.

8. The composition of claim 1 wherein at least about 0.1 weight percent of said particles are present.

9. The composition of claim 1 wherein at least about 0.5 weight percent of said particles are present.

10. A method for conditioning hair comprising applying an effective amount of the composition of claim 1 to the hair.

11. A hair conditioning composition comprising by weight:
(a) from about 0.1% to about 10.0% of an amidoamine having the following general formula:
R¹CONH(CH₂)ₘN(R²)₂
wherein R¹ is a fatty acid residue having from about 11 to about 24 carbon atoms, R² is an alkyl having from 1 to about 4 carbon atoms, and m is an integer from 1 to about 4;
(b) from about 0.01% to about 10.0% of an acid;
(c) from about 0.05% to about 20% of a high melting point compound having a melting point of at least about 25° C. selected from the group consisting of fatty alcohols, fatty acids, fatty alcohol derivatives, fatty acid derivatives, hydrocarbons, steroids, and mixtures thereof;
(d) from about 0.01% to about 10% particles; and
(e) water;
wherein the molar ratio of the amidoamine to the acid is from about 1:0.3 to about 1:1, and wherein the composition is substantially free of quaternary ammonium compounds.

12. The composition of claim 11 further comprising from about 0.02% to about 2% of an oily compound having a melting point of not more than about 25° C. selected from the group consisting of a first oily compound, a second oily compound, and mixtures thereof.

13. The hair conditioning composition according to claim 12 comprising by weight:
(a) from about 0.4% to about 5.0% of the amidoamine;
(b) from about 0.05% to about 2.0% of the acid;
(c) from about 1% to about 15% of the high melting point compound;
(d) from about 0.05% to about 10% particles;
(e) water; and
(f) from about 0.2% to about 2.0% of the oily compound.

14. The hair conditioning composition according to claim 12 comprising by weight:
(a) from about 1.0% to about 3.0% of the amidoamine;
(b) from about 0.3% to about 1.0% of the acid;
(c) from about 3% to about 8% of the high melting point compound;
(d) from about 0.1% to about 10% particles;
(e) water; and
(f) from about 0.2% to about 1.5% of the oily compound.

15. The hair conditioning composition according to claim 12 comprising by weight:
(f) from about 0.1 to about 0.75% of a first oily compound; and
(g) from about 0.1 to about 0.75% of a second oily compound.

16. The hair conditioning composition according to claim 15 wherein the first oily compound is selected from the group consisting of oleyl alcohol, palmitoleic alcohol, isostearyl alcohol, isocetyl alcohol, octyl dodecanol, octyl decanol, octyl alcohol, caprylic alcohol, decyl alcohol, lauryl alcohol, and mixtures thereof.

17. The hair conditioning composition according to claim 15 wherein the second oily compound is selected from the group consisting of mineral oil, isododecane, isohexadecane, polybutene, polyisobutene, and mixtures thereof.

18. The compositon of claim 11 wherein at least about 0.1 weight percent of said particles are present.

19. The composition of claim 11 wherein at least about 0.5 weight percent of said particles are present.

20. A method for conditioning hair comprising applying an effective amount of the composition of claim 11 to the hair.

21. A hair conditioning composition comprising by weight:
a) from about 0.1% to about 5% hollow particles;
b) from about 0.1% to about 10% of a cationic surfactant; and
c) water.

22. The composition of claim 21 comprising by weight:
a) from about 0.01% to about 10% hollow particles;
b) from about 0.25% to about 5% of a cationic surfactant; and
c) water.
